(19) United States
(12) Patent Application Publication
(10) Pub. No.: US 2009/0088831 A1
(43) Pub. Date: Apr. 2, 2009

(54) STENT AND STENT DELIVERY DEVICE

(75) Inventor: Hiroki GOTO, Tokyo (JP)

Correspondence Address: BUCHANAN, INGERSOLL & ROONEY PC, POST OFFICE BOX 1404, ALEXANDRIA, VA, 22313-1404, US

(73) Assignee: TERUMO KABUSHIKI KAISHA, Shibuya-ku (JP)

(21) Appl. No.: 12/239,272

(22) Filed: Sep. 26, 2008

(30) Foreign Application Priority Data
Sep. 28, 2007 (JP) 2007-254731

(51) Int. Cl. A61F 2/88

(52) U.S. Cl. 623/1.11; 623/1.43

(57) ABSTRACT

An stent includes an outer stent base layer formed from a metallic material, an inner stent base layer formed from a metallic material and positioned inside the outer stent base layer, and a resinous adhesive layer containing a biodegradable polymer which is placed between the outer stent base layer and the inner stent base layer, and bonds together the outer stent base layer and the inner stent base layer. The resinous adhesive layer contains a biodegradable polymer containing a physiologically active substance in such a manner as to permit it to release itself.

### TECHNOLOGICAL FIELD

The present invention generally relates to a stent and a stent delivery device for delivering the stent to a stenosed portion or closed portion of a lumen of an organism, such as a blood vessel, bile duct, trachea, esophagus, and urethra.

### BACKGROUND DISCUSSION

The stent is a medical tool or device, usually in a tubular form, used to treat various diseases caused by a stenosed or closed (narrowed or obstructed) part of a blood vessel or other intravital lumen. In use, the stent expands the narrowed or obstructed part and remains in place in the expanded part to keep the lumen open.

The stent has a small size (e.g., diameter) prior to placement in the living body to help facilitate insertion into the living body. The stent increases in diameter after it has been placed in the narrowed or obstructed part to keep the lumen open.

The stent is usually in the form of cylinder made from a metallic wire or tube. It is attached (in a reduced diameter state) to a catheter when it is inserted into a living body. After insertion into a living body, the stent is expanded in some way at the desired position so that it comes into close contact with the inside of the lumen and keeps the lumen open.

Generally speaking, stents fall into one of two classes of stents according to the stent's function and its manner of retention. The two classes are self-expandable stents and balloon expandable stents. The latter is not able to expand by itself. Rather, after being placed at the desired position, the stent is expanded (through plastic deformation) by a balloon introduced into it, so that it comes into close contact with the inside of the lumen. This type of stent requires a stent expansion step.

The stent is retained to at least reduce the possibility of, more preferably prevent, restenosis that might occur after an operation such as PTCA. Recently, this is achieved more effectively by employing a stent that carries a physiologically active substance which locally releases itself over an extended period of time at the desired position of the lumen where the stent is retained.

Japanese Patent Laid-open No. Hei-8-33718 (Patent Document 1) discloses a stent coated with a mixture of a therapeutic substance and a polymeric material. Japanese Patent Laid-open No. Hei-9-57807 (Patent Document 2) proposes a stent having thereon a layer of drug and a layer of biodegradable polymer, which are formed one over the other.

In addition, the present applicant has proposed new stents as disclosed in Japanese Patent Laid-open No. 2004-41704 (Patent Document 3), Japanese Patent Laid-open No. 2005-168937 (Patent Document 4) and Japanese Patent Laid-open No. 2006-87704 (Patent Document 5).

Metallic stents have long been in use because of their ability to effectively prevent restenosis. However, stents made of metal alone do not produce any additional effect, and hence they need a polymer coating containing a physiologically active substance for their additional effect.

The polymer-coated stents mentioned above are sufficiently effective, yet they have the possibility of causing inflammation because the polymer comes into direct contact with the blood vessel wall. Moreover, the exposed polymer layer is subject to breaking, peeling, and cracking when the stent is retained at the position of the stenosis. Moreover, the metallic stent body keeps the form which it takes when it is retained at the position of the stenosis and so it is possible that it could cause restenosis.

A conceivable way of addressing this is by forming the stent from a polymer material alone. However, polymer may not be sufficiently strong (radial force) to support the blood vessel. Also, polymer in an amount sufficient to support the blood vessel could cause inflammation, and the increased amount of polymer results in a thick wall which causes restenosis.

### SUMMARY

According to one aspect, a stent comprises an outer stent base layer of metallic material, an inner stent base layer of metallic material located inside the outer stent base layer, and a resinous adhesive layer between the outer stent base layer and the inner stent base layer. The resinous adhesive layer contains a biodegradable polymer and bonds together the outer stent base layer and the inner stent base layer. The resinous adhesive layer comprises a biodegradable polymer containing a physiologically active substance which is releasable.

According to another aspect, a stent comprises an outer stent base layer of metallic material, an inner stent base layer of metallic material located inside the outer stent base layer, and an intermediate stent base layer of metallic material positioned between the outer stent base layer and the inner stent base layer. A first resinous adhesive layer is between the outer stent base layer and the intermediate stent base layer, and the first resinous adhesive layer contains a biodegradable polymer and bonds together the outer stent base layer and the intermediate stent base layer. A second resinous adhesive layer is between the intermediate stent base layer and the inner stent base layer, and the second resinous adhesive layer contains a biodegradable polymer and bonds together the intermediate stent base layer and the inner stent base layer. The first resinous adhesive layer containing a physiologically active substance which is releasable.

The stent here has the inner and outer surfaces of metal and the adhesive layer which does not come into contact with the inside of the living body in which the stent is retained. Moreover, it is capable of releasing a physiologically active substance and becomes thin over a certain period of retention in a living body. The indwelling stent has a comparatively high degree of bioaffinity attributable to metal and is capable of releasing the physiologically active substance. In addition, because it becomes thinner after a certain period of indwelling in a living body, it is less susceptible to causing restenosis. Consequently, it is not likely to cause restenosis after retention in a living body.

According to another aspect, a stent delivery device comprises a tubular shaft body comprising a tip, a collapsible and expandable balloon attached to the tip of the tubular shaft body, and a stent such as described above, wherein the stent surrounds the balloon in a collapsed state and is expandable upon expansion of the balloon.

### BRIEF DESCRIPTION OF THE DRAWING FIGURES

FIG. 1 is a front view of one embodiment of the stent disclosed here.

FIG. 2 is a front view of the stent shown in FIG. 1 in a developmental state in which the stent is cut along its length and flattened.

FIG. 3 is an enlarged cross-sectional view taken along the section line A-A in FIG. 2.

FIG. 4 is an enlarged cross-sectional view of the linear part of a stent according to another example disclosed here.

FIG. 5 is an enlarged cross-sectional view of the linear part of a stent according to an additional example disclosed here.

FIG. 6 is an enlarged cross-sectional view of the linear part of an intravital stent according to another example disclosed here.

FIG. 7 is an enlarged cross-sectional view of the linear part of a stent according to a further example disclosed here.

FIG. 8 is an enlarged view of a portion of the stent shown in FIG. 2.

FIG. 9 is a development illustration showing the stent (shown in FIG. 1) being produced.

FIG. 10 is a development illustration showing a stent (in its manufacturing stage) according to another example disclosed here.

FIG. 11 is a front view of a stent delivery device according to one embodiment disclosed here.

FIG. 12 is an enlarged cross-sectional view of the tip portion of the stent delivery device shown in FIG. 11.

FIG. 13 is a front view, partially in cross-section, of a portion of the stent delivery device illustrating the function of the stent delivery device.

### DETAILED DESCRIPTION

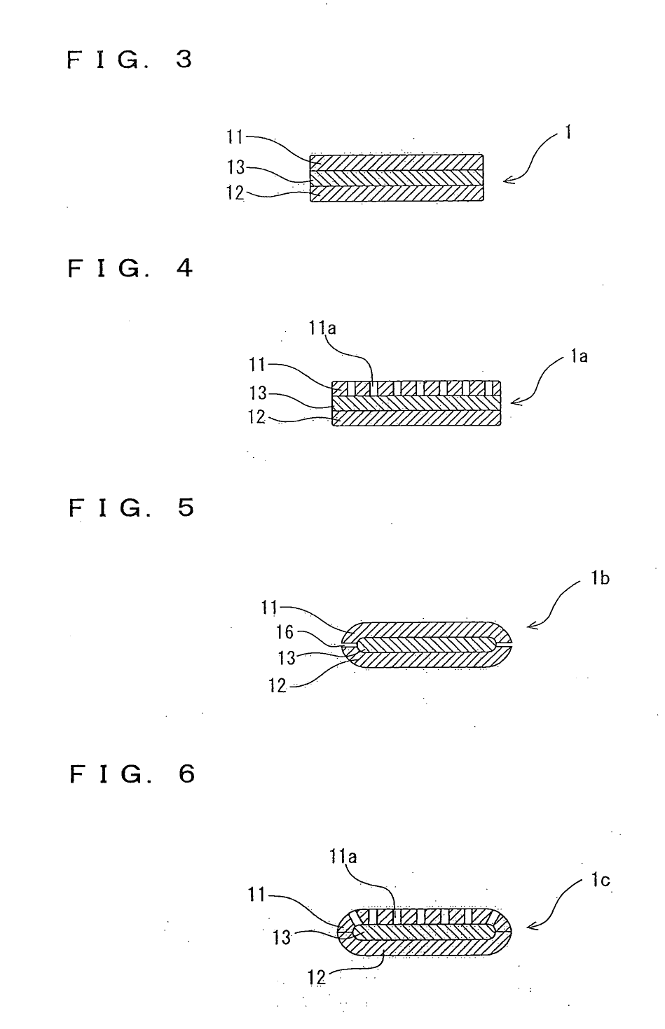

Referring initially to FIG. 3, the stent 1 according to one disclosed embodiment is composed of an outer base layer or outer stent base layer 11 made of a metallic material, an inner base layer or inner stent base layer 12 made of a metallic material, and an adhesive layer 13 interposed between the outer base layer 11 and the inner base layer 12 to bond them together. The adhesive layer 13 is made of a biodegradable polymer containing a physiologically active substance capable of being released.

According to this example, the cross-sectional shape of the stent 1 is defined by the outer layer 11 and the inner layer 12, both of which are made of a linear metallic material (wavy linear (strut) pattern). Both of the layers 11, 12 made of metallic materials possess a rectangular or plate-shaped cross-sectional shape. The two layers 11, 12 conform in shape to each other and so the outer base layer 11 and the inner base layer 12 have the same cross-sectional shape.

Thus, the stent 1 disclosed here has a multilayer structure composed of metallic outer and inner layers, with an adhesive layer interposed between them which is made of a biodegradable polymer.

The adhesive layer 13 is made of a biodegradable polymer so that it disappears (substantially disappears) after the biodegradable polymer has decomposed. After the adhesive layer 13 disappears, the stent retained in the living body becomes relatively thin and hence rarely causes restenosis.

The adhesive layer 13 may be one which still bonds the outer and inner layers 11 and 12 together even after decomposition of the biodegradable polymer constituting the adhesive layer 13. In this case, the adhesive layer may also contain a certain amount of non-biodegradable adhesive component. Despite the residual adhesive component, the stent becomes relatively thin after disappearance of the biodegradable polymer constituting the adhesive layer 13 and hence rarely causes restenosis.

The stent 1a shown in FIG. 4 may also be constructed so that the outer layer 11 is provided with a relatively large number of pores 11a (through-holes) extending from the outer surface of the layer 11 to the adhesive layer 13. This structure helps facilitate the release of the physiologically active substance contained in the adhesive layer 13.

FIG. 5 illustrates an alternative embodiment of the stent 1b in which the lateral edges of the outer layer 11 and the inner layer 12 are bent inward towards one another so that opposite edges of the adhesive layer 13 are covered in at least partial respects. This structure helps contribute to the stent keeping its shape relatively firmly. In the illustrated embodiment of FIG. 5, The edges of the outer layer 11 and the inner layer 12 are not in complete contact with each other so that a gap 16 remains between the facing edges of the layers 11, 12.

In the embodiment of the stent 1c shown in FIG. 6, the outer layer 11 is provided with a large number of pores (through-holes) 11a extending from the outer surface to the adhesive layer 13. In addition, the edges of the outer layer 11 and the inner layer 12 are bent inward. This structure helps contribute to the stent keeping its shape firmly. In addition, the edges of the outer layer 11 and the inner layer 13 may come into complete contact with each other as shown in FIG. 6. In this case, the physiologically active substance contained in the adhesive layer 13 releases itself through the pores 11a only, which is desirable from the standpoint of achieving a sustained release.

The stent 1 having the three-layered structure discussed above should have the dimensions specified below.

The thickness of the outer layer 11 is about 0.03 to 0.25 mm, preferably about 0.05 to 0.10 mm. The thickness of the inner layer 12 is about 0.03 to 0.25 mm, preferably about 0.05 to 0.10 mm. The thickness of the adhesive layer 13 is about 0.001 to 0.050 mm, preferably about 0.005 to 0.030 mm.

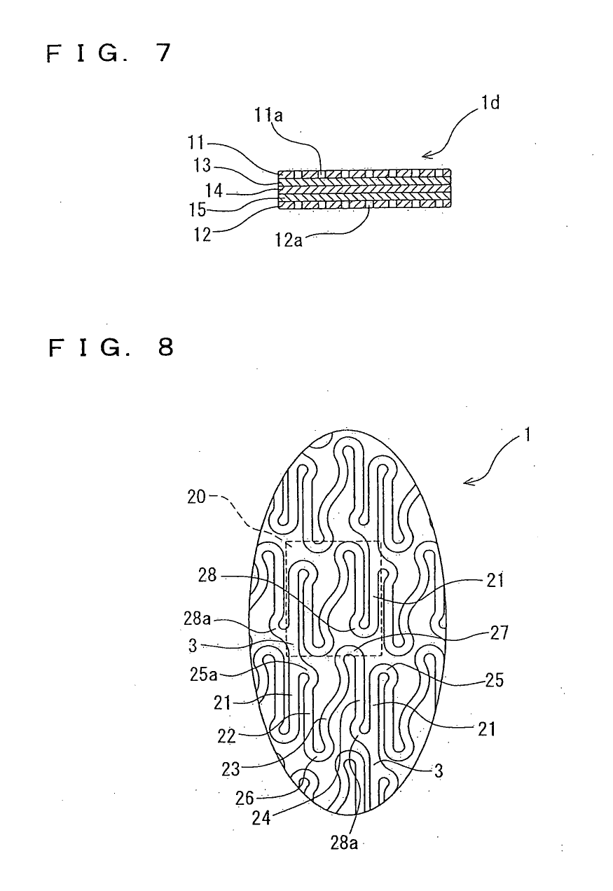

The multilayered structure of the stent can also take a form such as that shown in FIG. 7. Here, the stent 1d of multilayered structure is comprised of an outer layer 11 made of a metallic material, an inner layer 12 made of a metallic material, an intermediate layer 14 between the outer layer 11 and the inner layer 12, a first adhesive layer 13 made of a biodegradable polymer, and a second adhesive layer 15 made of a biodegradable polymer. The first adhesive layer 13 made of biodegradable polymer is positioned between the outer layer 11 and the intermediate layer 14 and bonds the two, layers 11, 14 together. The second adhesive layer 15 made of a biodegradable polymer is positioned between the inner layer 12 and the intermediate layer 14 and bonds the two layers 12, 14 together. The first adhesive layer 13 may contain a physiologically active substance that can be released from the layer.

The first adhesive layer 13 and the second adhesive layer 15 are made of a biodegradable polymer, so that they disappear (inclusive of substantially disappear) after the biodegradable polymer has decomposed.

The first adhesive layer 13 may continue bonding the outer and intermediate layers 11, 14 together even after decomposition of the biodegradable polymer constituting them, and the second adhesive layer 15 may continue bonding the inner and intermediate layers 12, 14 together even after decomposition of the biodegradable polymer constituting them. In this case, the adhesive layers may also contain a certain amount of non-biodegradable adhesive component.

The stent 1d according to this example shown in FIG. 7 is a five-layered structure composed of outer and inner metal layers, one intermediate metal layer, and two adhesive layers. Each adhesive layer is interposed between the intermediate metal layer and one of the inner and outer metal layers. However, the stent is not limited in this regard as it may have any other multi-layered structure. A typical multi-layered structure may be such that the inner layers, which are composed of metal layers and adhesive layers placed one over another, are surrounded by a metal layer.

The stent 1d according to this example is composed of the following layers. An outer stent base layer 11 is constructed as a linear body in stent form. An intermediate stent base layer 14 possesses a form conforming to the stent form of the outer stent base layer 11 and is positioned relative to the outer stent base layer in such a way that the stent form of the outer stent base layer 11 overlaps that of the intermediate stent base layer 14. Thus, the shape of the intermediate stent base layer 14 matches or is the same as the shape of the outer stent base layer 11.

An inner stent base layer 12 possesses a form conforming to the stent form of the intermediate stent base layer 14 and is positioned relative to the intermediate stent base layer 14 in such a way that the stent form of the intermediate stent base layer 14 overlaps that of the inner stent base layer 12. Thus, the shape of the intermediate stent base layer 14 matches or is the same as the shape of the inner stent base layer 12.

The stent 1d possessing a multilayered structure as shown in FIG. 7 may also be constructed such that the outer layer 11 has a large number of pores (through-holes) 11a extending from its outer surface to the first adhesive layer 13. This structure helps more easily release the physiologically active substance contained in the adhesive layer 13.

The second adhesive layer 15 should preferably contain a releasable physiologically active substance different from the releasable physiologically active substance contained in the first adhesive layer 13. The inner layer 12 may also have a large number of pores (through-holes) 12a extending from the surface to the second adhesive layer 15, as in the case of the stent 1d shown in FIG. 7. This structure helps more easily release the physiologically active substance contained in the adhesive layer 15.

The first and second adhesive layers 13, 15 should preferably be formed from a biodegradable material which contains a physiologically active substance for sustained release and continues bonding the outer layer 11, the inner layer 12, and the intermediate layer 14 even after decomposition of the biodegradable material.

The stent 1d possessing the five-layered structure described above and shown in FIG. 7 should have the dimensions specified below. The thickness of the outer layer 11 is about 0.03 to 0.15 mm, preferably about 0.05 to 0.10 mm. The thickness of the inner layer 12 is about 0.03 to 0.15 mm, preferably about 0.05 to 0.10 mm. The thickness of the intermediate layer 14 is about 0.03 to 0.15 mm, preferably 0.05 to 0.10 mm. The thickness of the first adhesive layer 13 is about 0.001 to 0.050 mm, preferably about 0.005 to 0.030 mm. The thickness of the second adhesive layer 15 is about 0.001 to 0.050 mm, preferably about 0.005 to 0.030 mm.

The stents disclosed herein should preferably be stents of the so-called balloon expandable type, which takes on an approximately tubular form, possesses a diameter adequate for insertion into the lumen of a living body, and expands upon application of an outward force in the radial direction.

The layers 11, 12, 14 of the stent should preferably be made of a metallic material with a certain degree of biocompatibility, such as stainless steel, tantalum or alloy thereof, platinum or alloy thereof, gold or alloy thereof, cobalt alloy, cobalt-chromium alloy, titanium alloy, and niobium alloy. The metallic material may undergo plating with noble metal (such as gold and platinum) after it has been formed into a stent. SUS316L stainless steel is most desirable for its good corrosion resistance qualities.

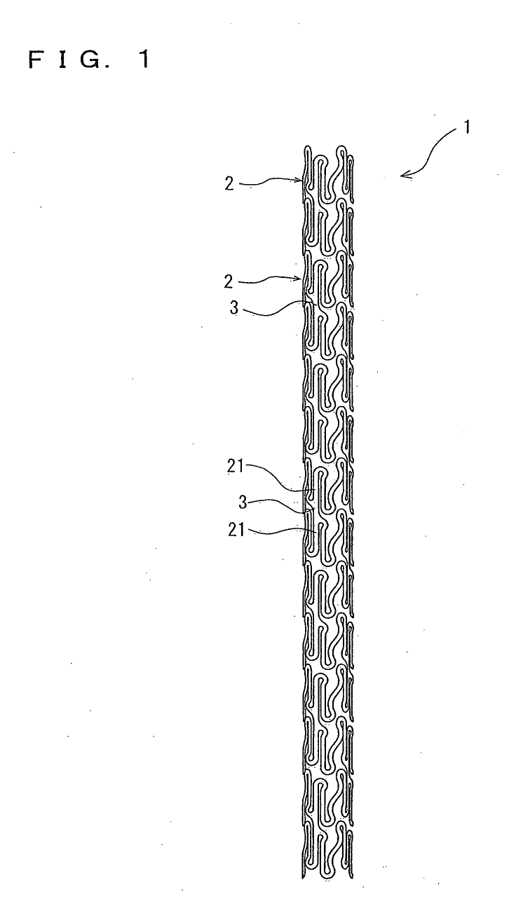

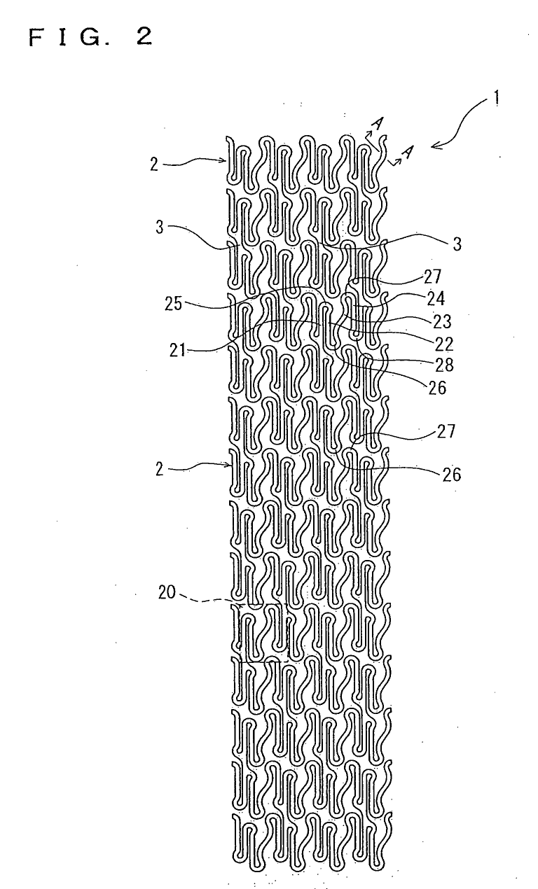

The stent before expansion should have a diameter of about 0.8 to 1.8 mm, preferably 0.9 to 1.6 mm. The stent before expansion should have a length of about 8 to 40 mm. As illustrated in FIGS. 1 and 2, the stent comprises a plurality of axially adjacent wavy annular members 2 disposed along the length of the stent. Each of the axially wavy annular members 2 should preferably have a length of about 1.0 to 2.5 mm.

The adhesive layers 13, 15 should preferably be formed from rubber, elastomer, or flexible resin. Preferred examples of rubber include silicone rubber and latex rubber. Preferred examples of elastomer include fluororesin elastomer, polyurethane elastomer, polyester elastomer, polyamide elastomer, and polyolefin elastomer (such as polyethylene elastomer and polypropylene elastomer). Preferred examples of flexible resin include polyurethane, polyester, polyamide, polyvinyl chloride, ethylene-vinyl acetate copolymer, and polyolefin (such as polyethylene, polypropylene, and ethylene-propylene copolymer). Of these examples, elastomer and rubber are most desirable. The desirable rubber is silicone rubber, particularly low-temperature curable or room-temperature curable silicone rubber.

As mentioned above, the adhesive layer contains a biodegradable material for sustained release of a physiologically active substance. The biodegradable material should be contained in an amount sufficient for the adhesive layer to continue bonding together the layers constituting the stent even after decomposition of the biodegradable material. The amount of the biodegradable material in the adhesive layer should be about 30 to 50 wt %, depending on the type of adhesive layer and the biodegradable material.

The biodegradable material is not specifically restricted so long as it is decomposed enzymatically or non-enzymatically in a living body into a nontoxic product. It includes, for example, polylactic acid, polyglycolic acid, polylactic acid-polyglycolic acid copolymer, polycaprolactone, polylactic acid-polycaprolactone copolymer, polyorthoester, polyphosphazene, polyphosphoric ester, polyhydroxylactic acid, polymalic acid, poly-α-amino acid, collagen, gelatin, laminin, heparan sulfate, fibronectin, vitronectin, condroitin sulfate, hyaluronic acid, polypeptide, chitin, and chitosan.

The physiologically active substance may be selected from any drug to suppress intimal thickening, anti-tumor agent, immunosuppressant, antibiotic, antirheumatic drug, antithrombotic drug, HMG-CoA reductase inhibitor, ACE inhibitor, calcium antagonist, antilipidemic agent, anti-inflammatory drug, integrin inhibitor, antiallergic drug, antioxidant, GPIIbIIIa antagonist, retinoid, flavonoid, carotinoid, lipid metabolism improver, DNA synthesis inhibitor, tyrosine kinase inhibitor, antiplatelet drug, vascular smooth muscle growth inhibitor, NO yield promoting substance, tissue-derived biomaterial, interferon, and epithelial cell obtained by gene engineering. The foregoing drugs may be used alone or in combination with one another.

Preferred examples of the anti-tumor agent include vincristine, vinblastine, vindesine, irinotecan, pirarubicin, paclitaxel, decetaxel, and methotrexate.

Preferred examples of the immunosuppressant include sirolimus, tacrolimus, azathioprine, cyclosporin, cyclophosphamide, mycophenolate mofetil, gusperimus, and mizoribine. Preferred examples of the antibiotics include mitomycin, adriamycin, doxorubicin, actinomycin, daunorubicin, idarubicin, pirarubicin, aclarubicin, epirubicin, peplomycin, and zinostatin stimalamer.

Preferred examples of the antirheumatic drug include methotrexate, sodium thiomalate, penicillamine, and lobenzarit. Preferred examples of the antithrombotic drug include heparin, aspirin, anti-thrombin drug, ticlopidine, and hirudin. Preferred examples of the HMG-CoA reductase inhibitor include cerivastatin, cerivastatin sodium, atorvastatin, nisvastatin, itavastatin, fluvastatin, fluvastatin sodium, simvastatin, lovastatin, and pravastatin. Preferred examples of the ACE inhibitor include quinapril, perindopril erubumine, trandolapril, cilazapril, temocapril, delapril, enalapril maleate, lisinopril, and captopril. Preferred examples of the calcium antagonist include nifedipine, nilvadipine, diltiazem, benidipine, and nisoldipine. Preferred examples of the antilipidemic agent include probucol. Preferred examples of the antiallergic drug include tranilast. Preferred examples of the retinoid include all-trans-retinoic acid. Preferred examples of the flavonoid and carotinoid include catechins (particularly epigallocatechin gallate), anthocyanin, proanthocyanidin, lycopene, and β-carotene. Preferred examples of the tyrosine kinase inhibitor include genistein, tyrphostin, and erbstatin. Preferred examples of the anti-inflammatory drug include steroid, such as dexamethazone and prednisolone. Preferred examples of the tissue-derived biomaterial include EGF (epidermal growth factor), VEGF (vascular endothelial growth factor), HGF (hepatocyte growth factor), PDGF (platelet derived growth factor), and bFGF (basic fibroblast growth factor).

In the case of the stent 1d described above by way of example, the first adhesive layer (positioned outside or facing toward the outside in use) may contain a physiologically active substance which is at least one species selected from anti-tumor agent, immunosuppressant, retinoid, flavonoid, DNA synthesis inhibitor, and tyrosine kinase inhibitor.

Also, the second adhesive layer (positioned inside or facing toward the inside in use) may contain a physiologically active substance which is at least one species selected from antibiotic, antirheumatic drug, antithrombotic drug, HMG-CoA reductase inhibitor, ACE inhibitor, calcium antagonist, antilipidemic agent, integrin inhibitor, antiallergic drug, antioxidant, GPIIbIIIa antagonist, carotinoid, lipid metabolism improver, antiplatelet drug, anti-inflammatory drug, tissue-derived biomaterial, interferon, and NO formation promoter.

The metallic layer used in the stent disclosed here may have its surface (facing the adhesive layer) entirely or partly pretreated for good adhesion with the adhesive layer. A preferred method for pretreatment is by surface coating with a primer having a high degree of affinity. A variety of primers may be used. The most desirable one is a silane coupling agent which has hydrolyzable groups and organic functional groups. The hydrolyzable groups (such as alkoxyl groups) of the silane coupling agent decompose to form silanol groups which combine (through covalent bonding) with the metallic layer constituting the stent. The organic functional groups (such as epoxy group, amino group, mercapto group, vinyl group, and methacryloxy group) of the silane coupling agent chemically combine with the polymer constituting the adhesive layer. Typical examples of the silane coupling agent include γ-aminopropylethoxysilane and γ-glycidoxypropyldimethoxy-silane. Other primers than silane coupling agents include, for example, organotitanium coupling agent, aluminum coupling agent, chromium coupling agent, organophosphate coupling agent, organic vapor deposition film such as polyparaxylene, cyanoacrylate adhesive, and polyurethane paste resin.

The stent disclosed here may be of the so-called self-expanding type, which decreases in diameter at the time of insertion and restores or returns to an increased diameter state (increases in diameter) at the time of release in a living body.

In this case, the metallic layer constituting the stent should preferably be made of superelastic metal or superelastic alloy. Superelastic alloy is a synonym for shape memory alloy. It exhibits superelasticity at the temperature of a living body (about 37° C.). It includes Ti—Ni alloy (containing 49 to 53 at % Ni), Cu—Zu alloy (containing 38.5 to 41.5 wt % Zn), Cu—Zn—X alloy (containing 1 to 10 wt % X=Be, Si, Sn, Al, or Ga), and Ni—Al alloy (containing 36 to 38 at % Al). Of these alloys, Ti—Ni alloy is most desirable. The Ti—Ni alloy may be modified by incorporation with 0.01 to 10.0 at % X (where X is Co, Fe, Mn, Cr, V, Al, Nb, W, or B). The Ti—Ni alloy may also be modified by incorporation with 0.01 to 30.0 at % X (where X is Cu, Pb, and Zr). These alloys may have their mechanical properties changed as desired by properly selecting the ratio of cold working and/or the condition of final heat treatment. The superelastic alloy to be used for the stent should have a buckling strength (yield stress under loading) of 5 to 200 kg/mm², preferably 8 to 150 kg/mm², at 22° C., and a restoration stress (yield stress without loading) of 3 to 180 kg/mm², preferably 5 to 130 kg/mm², at 22° C. The term “superelasticity” means that when the alloy is deformed (bent, stretched, or compressed) to a region where ordinary metal undergoes plastic deformation at the temperature of use, it returns to or restores its original shape without requiring heating as soon as it is relieved from stress.

A way of manufacturing or producing the stent disclosed here involves preparing a first metal tube having a prescribed inside diameter and a second metal tube having a prescribed outside diameter which is slightly smaller than the inside diameter of the first metal tube. These two metal tubes are used to produce two stent base layers, which are similar in shape but different in diameter, by removing that part of each metal tube which does not constitute the stent base layer. This step is accomplished by photofabrication (or chemical etching through a mask), electric discharge machining, or cutting (such as mechanical grinding and laser cutting). The thus prepared stent base layers should preferably have their edges smoothened by chemical polishing or electrolytic polishing.

The outer surface of the stent base layer having a smaller diameter is subsequently coated with an adhesive material containing a physiologically active substance (preferably containing also a biodegradable material). Before the adhesive material is cured, the stent base layer having the larger diameter is slipped on and pressed against the coated adhesive material. In this way, there is obtained the desired stent. The stent base layer having the larger diameter will have a large number of pores if it is made from a porous metal tube.

The method of producing the stent is not restricted to the method described above. Another possible method may involve preparing the first metal tube having a prescribed inside diameter, and a second metal tube having a prescribed outside diameter which is slightly smaller than the inside diameter of the first metal tube. The second metal tube is coated with an adhesive material (preferably containing a biodegradable material). Before the adhesive material is cured, the first metal tube is slipped on and pressed against the second metal tube. Thus there is obtained a multi-layered tube having outer and inner metal layers. That part of the multi-layered tube which does not constitute the stent is removed. This step may be accomplished by any of the above-mentioned processes. The resulting intermediate product is dipped in a solution containing a physiologically active substance, so that the adhesive material supports (by migration or adsorption) the physiologically active substance. In this way there is obtained the stent as desired. The solution containing a physiologically active substance may be one which swells or slightly dissolves the adhesive material. In this case, the stent base layer having a larger diameter should preferably be made from a porous metal tube. This allows the adhesive material to support the physiologically active substance in a larger amount. The above-mentioned manufacturing processes may include a step of primer coating which enhances adhesion between the adhesive layer and the metal tube or the stent base layer.

The stent of the present invention may take on any configuration formed by the linear body.

As described above, the stent 1 shown in FIG. 1 is constructed of wavy annular elements or units 2 juxtaposed in the stent's axial direction. The axially adjacent wavy annular elements 2 are connected to each other such that they form a tubular body which has a diameter small enough for insertion into the lumen of a living body. The tubular body is expandable upon application of a force in its radial direction from its inside. Each wavy annular element 2 has at least one straight part 21 which extends parallel to the central axis of the stent 1 before and after the stent 1 is expanded. The straight part 21 of each wavy annular element 2 is connected at its connection part 3 to the straight part 21 of the axially adjacent annular element 2. More specifically, in the illustrated embodiment shown in FIG. 2, each of the wavy annular elements, except the two axial end-most wavy annular elements, includes a total of four straight parts 21, two of which are connected to the respective straight part of the wavy annular element on one axial end, and the other two of which are connected to respective straight parts of the wavy annular element on the other axial end. The axial end-most wavy annular elements each include two straight parts 21 connected to respective straight parts of the axially adjacent wavy annular element.

This stent 1 is of so-called balloon expandable type, which is in a tubular shape having a diameter small enough for insertion into a living body and which is expandable upon application of a force in the radial direction from its inside.

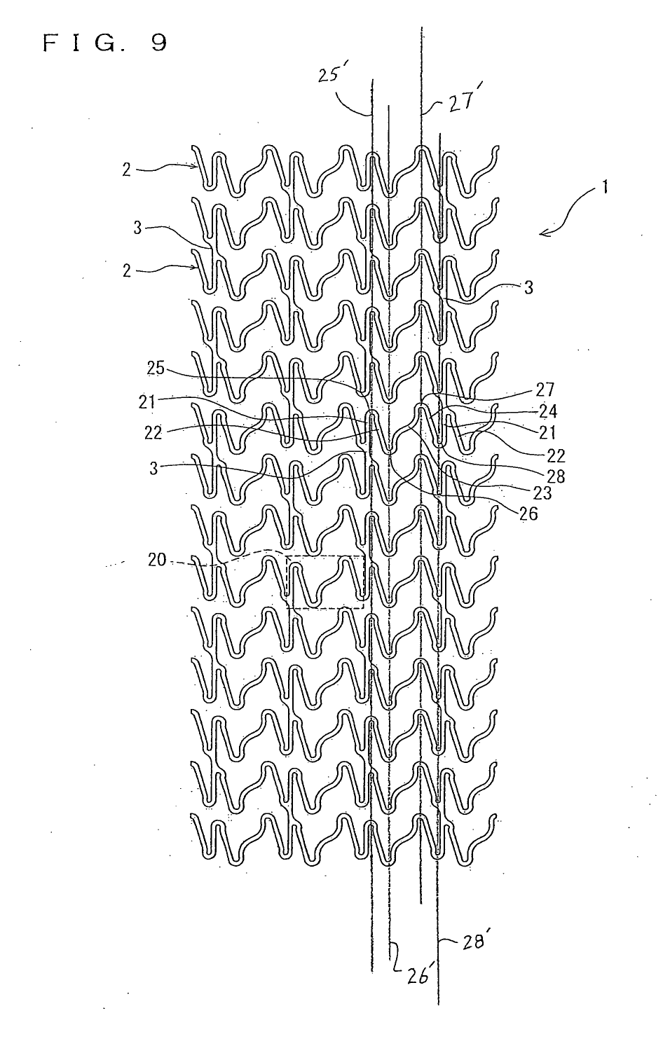

As described above, the stent 1 disclosed here, as shown in FIGS. 1 and 2, is composed of a plurality of wavy annular elements 2 which are juxtaposed in the axial direction and connected with one another. The number of wavy annular elements is fourteen (14) in the case of the stent shown in FIGS. 1 and 2 (and FIGS. 8 and 9). The number of wavy annular elements forming the stent may vary from 4 to 50, preferably 10 to 35, depending on the length of the stent.

Each wavy annular element 2 is comprised of an endless circular wavy linear body (wavy linear (strut) pattern), each having upwardly bent parts 25 and 27 and downwardly bent parts 26 and 28 which are formed alternately in the same number. In other words, the number of upwardly bent parts 25, 27 in each wavy annular element is the same as the number of downwardly bent parts 26, 28 in the wavy annular element.

As shown in FIGS. 1 and 2 (and 8 and 9), the wavy annular element 2 is composed of a plurality of units 20 (each taking on a shape of deformed letter M) which are connected to one another. Each unit 20 is composed of the following four linear parts: a straight part 21 (parallel to the axis of the stent); a first oblique straight part 22 which is connected to one end of the parallel straight part 21 through a bent part 25 (25a) and which becomes oblique at a prescribed angle with respect to the central axis of the stent 1 when the stent 1 is expanded; an oblique curved part 23 which is connected to one end of the first oblique straight part 22 through a bent part 26 and which extends at a prescribed angle with respect to the central axis of the stent 1; and a second oblique straight part 24 which is connected to one end of the oblique curved part 23 through a bent part 27 and which becomes oblique at a prescribed angle with respect to the central axis of the stent 1 when the stent 1 is expanded. The adjacent units 20 are connected to each other through the bent part 28 (28a) which connects one end of the second oblique straight part 24 with one end of the parallel straight part 21, so that they form the endless wavy annular element 2. This structure helps prevent the wavy annular element 2 from shortening in the axial direction when the stent is expanded and also imparts a sufficient expanding force to the wavy annular element 2.

In the stent 1 of this example, the bent parts 25 are arranged approximately on straight lines parallel to the axial direction (axis) of the stent. In other words, a straight line connects the same point on the commonly positioned bent part 25 of each wavy annular element, and this straight line is substantially parallel to the axis of the stent. One example of a straight line passing through the same point of commonly positioned bent parts 25 of the wavy annular elements is identified as 25′ in FIG. 9. Similarly, the bent parts 27 are arranged approximately on straight lines parallel to the axial direction (axis) of the stent such that a straight line 27′ connects the same point on the commonly positioned bent part 27 of each wavy annular element, and this straight line 27′ is substantially parallel to the axis of the stent. Further, the bent parts 26, 28 are arranged approximately on straight lines parallel to the axial direction (axis) of the stent such that a straight line 26′, 28′ connects the same point on the commonly positioned bent part 26, 28 of each wavy annular element, and these straight lines 26′, 28′ are each substantially parallel to the axis of the stent.

The adjacent wavy annular elements 2 are connected to one another by the connecting part 3. In the stent 1 of this example, the ends of the parallel straight parts 21 of the axially adjacent wavy annular elements 2 are connected by the proximate short connecting part 3. This structure reduces the distance between the adjacent wavy annular elements 2 and also prevents the adjacent wavy annular elements 2 from forming between them any part which lacks the expanding force.

In the stent 1 of this example as shown in FIGS. 1 and 2 (and FIGS. 8 and 9), two of the parallel straight parts 21 which are connected to each other by the connecting part 3 are linear. This structure helps prevent the stent from shortening between the adjacent wavy annular elements when the stent is expanded. In addition, the stent 1 has a plurality of connecting parts 3 connecting the adjacent wavy annular elements to one another. This structure prevents the adjacent wavy annular elements from separating and imparts a sufficient expanding force to the stent entirely.

The stent of this example does not have any part which is composed of more than two parallel straight parts 21 joined together in the axial direction by the connecting part. In other words, the stent is constructed such that it has only two parallel straight parts 21 which are joined together, but does not have three parallel straight parts 21 joined together (the connecting parts 3 connect only two straight parts 21 from different wavy annular elements). This structure helps prevent deformation in one wavy annular element (conforming to the blood vessel) from spreading directly to another one through its adjoining one. Thus this structure permits individual wavy annular elements to perform their expanding functions individually.

As mentioned above, the stent 1 has a plurality of connecting parts 3 joining together axially adjoining wavy annular elements 2. Thus the connecting parts 3 help prevent adjoining wavy annular elements 2 from separating from each other unnecessarily, and the stent as a whole exhibits its expanding force sufficiently. As an alternative to the illustrated embodiment shown in FIGS. 1 and 2, there may be only one connecting part 3 between axially adjoining wavy annular elements. The connecting part 3 should have a length (in the axial direction of the stent 1) shorter than about 1.0 mm, preferably 0.1 to 0.4 mm.

The stent 1 of this example has two connecting parts 3 which join together axially adjoining wavy annular elements 2, and the connecting parts 3 are arranged diametrically opposite to each other. The connecting parts 3 are arranged such that they are not contiguous in the axial direction of the stent 1. To be specific, in the case of the stent 1 of this example (shown in FIGS. 1, 2, 8 and 9), a first pair of connecting parts 3 connecting first and second axially adjacent wavy annular elements are arranged diametrically opposite to each other, and a second pair of connecting parts 3 connecting the second wavy annular elements and a third wavy annular element that is axially adjacent the second wavy annular element are also arranged diametrically opposite to each other, but the second pair of connecting parts 3 is rotationally displaced by about 90° (around the central axis of the stent 1) from the first pair.

The stent 1 is formed, with its outside diameter larger than that shown in FIGS. 1 and 2. Then, it is mounted on an expandable balloon and made to shrink in the radial direction. Thus, the stent 1 is expanded as the balloon is expanded.

The stent 1 in its unexpanded state should have a diameter of about 0.8 to 1.8 mm, particularly 0.9 to 1.6 mm, and also have a length of about 8 to 40 mm. Each wavy annular element 2 should preferably be about 1.0 to 2.5 mm long (i.e., the axial length of one wavy annular element or the length of one wavy annular element measured along the axis of the stent is preferably about 1.0 to 2.5 mm.

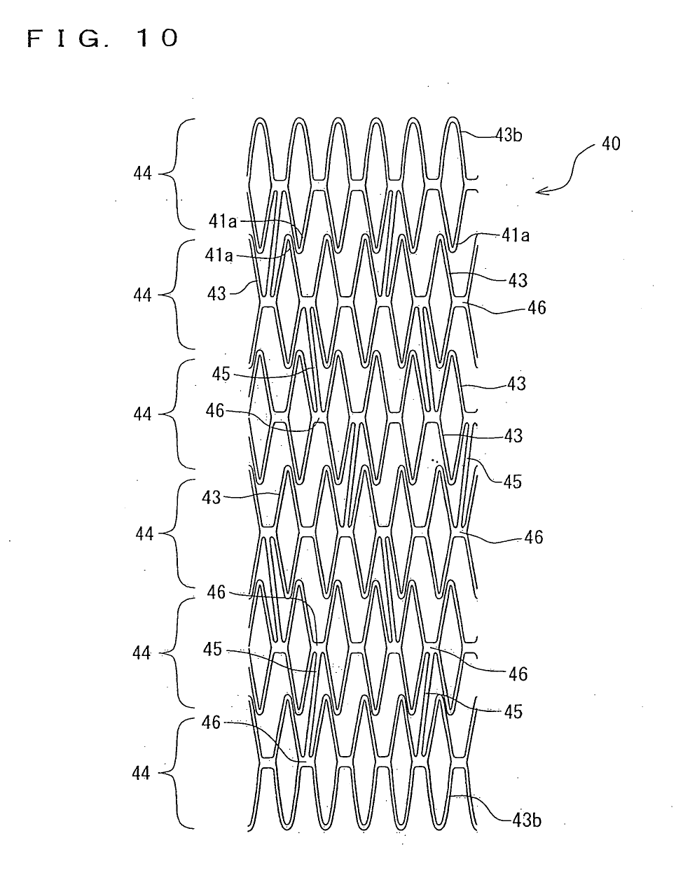

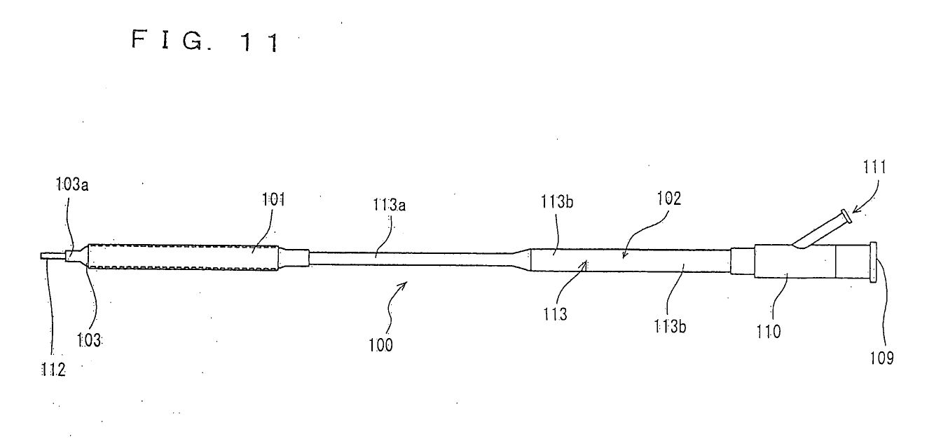

The stent disclosed here may take on any shape (frame form constructed of linear members) other than that described above and shown in the drawing figures mentioned above. One different shape is shown in FIG. 10. In this case the wavy annular elements are replaced by another annular element comprised of a plurality of ring linear bodies 43, which is expandable upon application of a force in the radial direction.

The stent 40 of this example is formed in a cylindrical shape having a diameter small enough for insertion into the lumen of a living body, and it is capable of expansion upon application of an outward force in the radial direction. The stent 40 includes a plurality of axially adjacent annular elements or units 44 joined to one another consecutively so that the annular elements 44 are arranged in the axial direction. Each annular unit 44 is composed of a plurality of circumferentially adjacent ring-shaped linear bodies 43, comprised of a plurality of bends and openings, which expand upon application of a force in the radial direction. The stent 40 also has connecting parts 45 which join together the axially adjoining annular units 44. In this illustrated embodiment, There are no connecting parts 45 contiguous in the axial direction of the stent 40. More than one connecting part 45 extends between axially adjoining annular units, and they are also arranged diametrically opposite to each other and at equal intervals around the central axis of the stent 40.

The stent 40 shown in FIG. 10 is composed of the annular units 44 which are arranged in the axial direction thereof. Each annular unit 44 is composed of ring-shaped linear bodies 43, each having bends 41a (which extend in the axial direction of the stent 40) and an opening. The ring-shaped linear bodies 43 are arranged at nearly equal intervals along the circumference of the stent 40 and are joined together in the circumferential direction by the connecting parts 46. One annular unit 44 is joined to its axially adjacent annular units by at least two connecting parts 45. The stent 40 may be envisaged as a tubular body consisting of a large number of annular units 44 joined together by the connecting parts 45.

According to this example, the annular unit 44 consists of six ring-shaped linear bodies 43 which are arranged at nearly equal angular intervals. Each linear ring 43 is elongated in the axial direction of the stent 40 so that it resembles a rhombus or a diamond. The center of each ring-shaped linear body 43 has a rhombic opening. The bends 43b constitute both ends (in the axial direction) of the stent. Each ring-shaped linear body 43 is configured so that it is closed and entirely surrounds the opening that opens on the side of the stent. This structure helps allow the stent to keep a strong expanding force. In addition, each ring-shaped linear body 43 is curved in the circumferential direction so that they are arranged at nearly equal intervals.

Each ring-shaped linear body 43 is joined to the adjoining body (in the circumferential direction) by the connecting parts 46, which are positioned at the sides of each ring-shaped linear body 43. In other words, the ring-shaped linear bodies 43 are joined together in the circumferential direction by the connection parts 46. The connecting parts 46 remain substantially at the same positions when the stent 40 is expanded, and the structure helps allow the expanding force to be applied to the center of the ring linear body 43, with the result that all the ring linear bodies 43 uniformly expand (deform).

The connecting part 46 of one annular unit 44 is joined to the connecting part 46 of its axially adjoining annular unit 44 by the linking part 45, which is slightly longer (as compared with the connecting part) and parallel to the axial direction of the stent 40. To be more specific, the axially adjacent annular units 44 are joined together by the linking parts 45 which link the connecting parts 46. The annular units 44 at both ends of the stent 40 have the ring-shaped linear bodies 43 whose outer parts 43b are nearly elliptic.

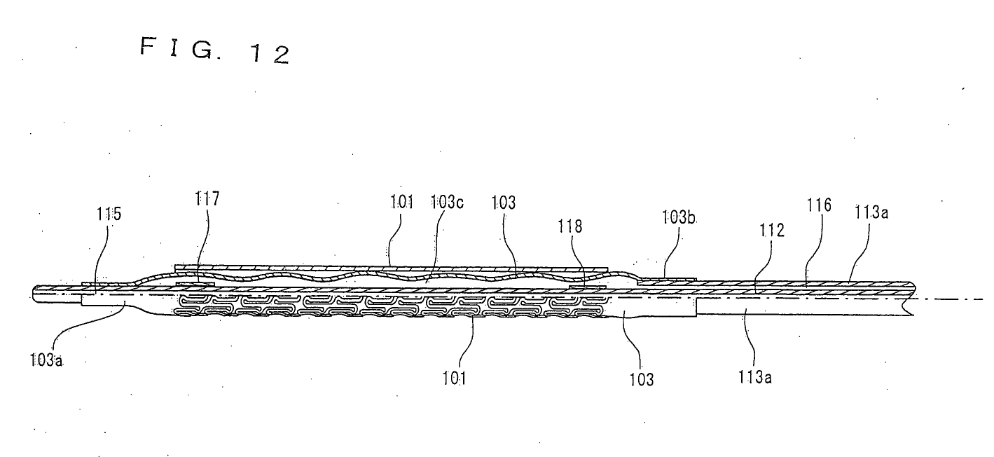

The stent delivery device disclosed here will now be described below with reference to an example shown in the accompanying FIGS. 11-13.

The stent delivery device 100 according to one disclosed embodiment includes a tubular shaft body 102, a collapsible and expandable balloon 103 attached to the tip of the shaft body 102, and a stent 101 which surrounds the collapsed balloon 103 and which can be expanded by the balloon 103.

The stent 101 may be a stent such as the stent 1 described above. The stent has a diameter small enough for insertion into the lumen of a living body, and it is of the so-called balloon expandable type which can be expanded upon application of an outward force in the radial direction. The desirable stent used for this purpose should be composed of linear members which account for 60% to 80% of the external area (including voids) of the stent placed on the balloon 103. The shaft body 102 has a balloon expanding lumen, one end of which communicates with the balloon. Also, the shaft body 102 has one or more X-ray opaque objects fixed to its outside. One of them is placed at the center of the stent, and two of them are placed respectively at positions a certain distance away from the center of the stent.

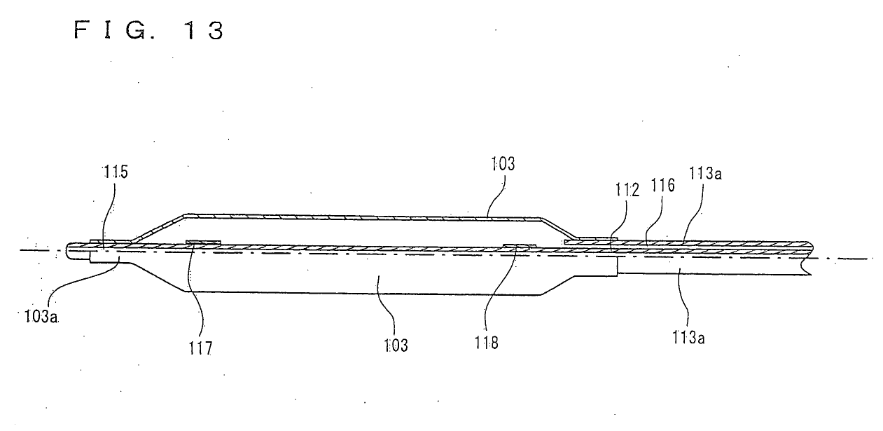

As shown in FIG. 12, the shaft body 102 is provided with a guide wire lumen 115, one end of which opens at the forward end of the shaft body 102 and the other end of which opens at the rear end of the shaft body 102.

The stent delivery device 100 has the shaft body 102, the stent expanding balloon 103 attached to the forward end of the shaft body 102, and the stent 101 mounted on the balloon 103. The shaft body 102 is comprised of the inner tube 112, the outer tube 113, and the branching hub 110.

The inner tube 112 has the guide wire lumen 115 for the guide wire to pass through it, as shown in FIG. 12. It has a length of 100 to 2500 mm, preferably 250 to 2000 mm, an outside diameter of 0.1 to 1.0 mm, preferably 0.3 to 0.7 mm, and a wall thickness of 10 to 250 μm, preferably 20 to 100 μm. It is passed through the outer tube 113 such that its forward end projects from the outer tube 113. The balloon expanding lumen 116 is formed between the outer surface of the inner tube 112 and the inner surface of the outer tube 113. It has a sufficient space. The outer tube 113 holds therein the inner tube 112 passing through it, and its forward end is at a position slightly behind the forward end of the inner tube 112.

The outer tube 113 has a length of 100 to 2500 mm, preferably 250 to 2000 mm, an outside diameter of 0.5 to 1.5 mm, preferably 0.7 to 1.1 mm, and a wall thickness of 25 to 200 μm, preferably 50 to 100 μm.

The outer tube 113 consists of the forward part 113a and the rear part 113b (close to the shaft body 102), which are joined together. The forward part 113a tapers off.

The forward end of the forward part 113a has an outside diameter of 0.50 to 1.5 mm, preferably 0.60 to 1.1 mm, and the rear end of the forward part 113a and the outer tube 113b close to the shaft body 102 have an outside diameter of 0.75 to 1.5 mm, preferably 0.9 to 1.1 mm.

The balloon 103 has the forward connecting part 103a and the rear connecting part 103b. The forward connecting part 103a is fixed at a position slightly behind the forward end of the inner tube 112. The rear connecting part 103b is fixed to the forward end on the outer tube 113. Also, the balloon 103 communicates with the balloon expanding lumen 116 near the base end.

The inner tube 112 and the outer tube 113 should preferably be formed from a moderately flexible material, such as thermoplastic resins, silicone rubber, and latex rubber. Thermoplastic resins are desirable, and they include polyolefins (such as polyethylene, polypropylene, ethylene-propylene copolymer, and ethylene-vinyl acetate copolymer), polyvinyl chloride, polyamide elastomer, and polyurethane. Of these examples, polyolefins are most desirable.

The balloon 103 is collapsible as shown in FIG. 12. In its unexpanded state, the balloon 103 can be folded over or placed on the outer surface of the inner tube 112. In its expanded state, the balloon 103 has a cylindrical part (tubular expandable part) which has almost the same diameter as that of the stent 101 mounted thereon, so that the balloon 103 can expand the stent 101. The cylindrical part does not necessarily need to be a true cylinder; it may be a polygonal column. The balloon 103 has its forward end 103a bonded to the inner tube 112 and its rear end bonded to the outer tube 113 by an adhesive or by heat fusion bonding to ensure fluid-tightness, as mentioned above. In addition, the balloon 103 has a tapering intermediate part between the expandable part and the bonding part.

The balloon 103 has an expanding space 103c between its inside and the outside of the inner tube 112. The expanding space 103c communicates (through the entire circumference) at its rear end with the expanding lumen 116. Since the rear end of the balloon 103 communicates with the expanding lumen having a comparative large volume, it is possible to surely inject the expanding fluid into the balloon through the expanding lumen 116.

The balloon 103 should preferably be formed from a moderately flexible material, such as thermoplastic resins, silicone rubber, and latex rubber. Thermoplastic resins are desirable, and they include polyolefins (such as polyethylene, polypropylene, ethylene-propylene copolymer, ethylene-vinyl acetate copolymer, and crosslinked ethylene-vinyl acetate copolymer), polyvinyl chloride, polyamide elastomer, polyurethane, polyesters (such as polyethylene terephthalate), polyarylene sulfide (such as polyphenylene sulfide). Preferable among them is extensible one capable of orientation. The balloon 103 should preferably be made of a biaxially oriented material having a high strength and a high degree of expansion.

The balloon 103 is specified by the following dimensions. The cylindrical part (or the expandable part) in its expanded state should have an outside diameter of 2 to 4 mm, preferably 2.5 to 3.5 mm, and a length of 10 to 50 mm, preferably 20 to 40 mm. The forward bonding part 103a should have an outside diameter of 0.9 to 1.5 mm, preferably 1 to 1.3 mm, and a length of 1 to 5 mm, preferably 1 to 1.3 mm. The rear bonding part 103b should have an outside diameter of 1 to 1.6 mm, preferably 1.1 to 1.5 mm, and a length of 1 to 5 mm, preferably 2 to 4 mm.

The stent delivery device 100 has two X-ray opaque members 117 and 118, as shown in FIG. 12. They are fixed respectively to the outside of the shaft body 112 at spaced apart positions, namely both ends of the expanded cylindrical part or the expandable part. They may also be fixed to the outside of the shaft body 102 (or the inner tube 112 in this example) at positions a certain distance away from the center of the stent 101. Alternatively, one X-ray opaque object may be fixed to the outside of the shaft body at the center of the stent.

The X-ray opaque members 117, 118 may be in the form of ring or coil of wire. It is preferable that X-ray opaque members are made of gold, platinum, tungsten, alloys thereof, and silver-palladium alloy.

The stent 101 is mounted on the balloon 103, with the stent covering the folded balloon 103. The stent 101 is formed from a metallic tube having a smaller diameter than the expanded stent and an inside diameter larger than the outside diameter of the collapsed balloon. With the balloon inserted therein, the stent has its diameter reduced by application of a uniform inward force. In this way there is obtained the stent ready for use. In other words, the above-mentioned stent 101 is completed when it is slipped on and pressed against the balloon inserted therein.

A linear reinforcement member may be inserted between the inner tube 112 and outer tube 113, namely into the balloon expansion lumen 116. The stiffness imparting object prevents the main body 102 of the stent delivery device 100 from extremely bending without excessively decreasing its flexibility. It also facilitates insertion of the forward end of the stent delivery device 100. It should preferably have its diameter reduced by grinding at its forward end. Moreover, it should preferably extend to the vicinity of the forward end of the outer tube 113 of the main body. A desirable reinforcement member may be a metallic wire having a diameter of 0.05 to 1.50 mm, preferably 0.10 to 1.00 mm. The metallic wire should preferably be that of elastic metal or superelastic metal, such as stainless steel, particularly high strength stainless steel for spring and superelastic alloy.

The stent delivery device 100 of this example has the branching hub 110 fixed to the base end, as shown in FIG. 11. The branching hub 110 has the guide wire port 109 that communicates with the guide wire lumen 115. It also has the inner tube hub fixed to the inner tube 112 and the outer tube hub fixed to the outer tube 113. The outer tube hub 113 communicates with the balloon expanding lumen 116 and has the injection port 111. The outer tube hub and the inner tube hub are fixed to each other. The branching hub 110 may be formed from a thermoplastic resin such as polycarbonate, polyamide, polysulfone, polyarylate, and methacrylate-butylene-styrene copolymer.

The stent delivery device 100 is not restricted in structure to the one mentioned above. It may have at its intermediate part the guide wire port that communicates with the guide wire lumen.

The principles, embodiments and modes of operation of the apparatus have been described in the foregoing specification, but the invention which is intended to be protected is not to be construed as limited to the particular embodiments of the apparatus disclosed. The embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is clamed is:

1. A stent comprising:
an outer stent base layer of metallic material;
an inner stent base layer of metallic material located inside the outer stent base layer; and
a resinous adhesive layer between the outer stent base layer and the inner stent base layer, the resinous adhesive layer containing a biodegradable polymer and bonding together the outer stent base layer and the inner stent base layer;
the resinous adhesive layer comprising a biodegradable polymer containing a physiologically active substance which is releasable.

2. The stent as defined in claim 1, wherein:
the outer stent base layer is a linear body in a form of a stent;
the inner stent base layer possesses a form conforming to the stent form of the outer stent base layer; and
the inner stent base layer and the outer stent base layer overlapping one another.

3. The stent as defined in claim 1, wherein the resinous adhesive layer is formed from the biodegradable polymer such that it disappears after decomposition of the biodegradable polymer.

4. The stent as defined in claim 1, wherein the resinous adhesive layer continues to bond together the outer stent base layer and the inner stent base layer even after decomposition of the biodegradable polymer.

5. The stent as defined in claim 1, wherein the inner stent base layer comprises a plurality of through holes extending from an inner surface of the inner stent base layer to the resinous adhesive layer.

6. The stent as defined in claim 1, wherein the outer stent base layer comprises a plurality of through holes extending from an outer surface of the outer stent base layer to the resinous adhesive layer.

7. The stent as defined in claim 1, wherein the physiologically active substance is at least one species selected from the group consisting of anti-tumor agent, immunosuppressant, antibiotic, antirheumatic drug, antithrombotic drug, HMG-CoA reductase inhibitor, ACE inhibitor, calcium antagonist, antilipidemic agent, integrin inhibitor, antiallergic drug, antioxidant, GPIIbIIIa antagonist, retinoid, flavonoid, carotinoid, lipid metabolism improver, DNA synthesis inhibitor, tyrosine kinase inhibitor, antiplatelet drug, anti-inflammatory drug, tissue-derived biomaterial, interferon, and NO yield promoting substance.

8. A stent comprising:
an outer stent base layer of metallic material;
an inner stent base layer of metallic material located inside the outer stent base layer;
an intermediate stent base layer of metallic material positioned between the outer stent base layer and the inner stent base layer;
a first resinous adhesive layer between the outer stent base layer and the intermediate stent base layer, the first resinous adhesive layer containing a biodegradable polymer and bonding together the outer stent base layer and the intermediate stent base layer;
a second resinous adhesive layer between the intermediate stent base layer and the inner stent base layer, the second resinous adhesive layer containing a biodegradable polymer and bonding together the intermediate stent base layer and the inner stent base layer; and
the first resinous adhesive layer containing a physiologically active substance which is releasable.

9. The stent as defined in claim 8, wherein the second resinous adhesive layer contains a physiologically active substance different from the physiologically active substance in the first resinous adhesive, the physiologically active substance in the second resinous adhesive layer being releasable.

10. The stent as defined in claim 8, wherein:
the outer stent base layer is constructed as a linear body in stent form,
the intermediate stent base layer possesses a form conforming to the stent form of the outer stent base layer and is positioned relative to the outer stent base layer in such a way that the stent form of the outer stent base layer is the same as that of the intermediate stent base layer, and
the inner stent base layer possessing a form conforming to the stent form of the intermediate stent base layer and is positioned relative to the intermediate stent base layer in such a way that the stent form of the intermediate stent base layer overlaps that of the inner stent base layer.

11. The stent as defined in claim 8, wherein the first and second resinous adhesive layers are formed from a biodegradable polymer such that they disappear after decomposition of the biodegradable polymer.

12. The stent as defined in claim 8, wherein the first and second resinous adhesive layers continue bonding together the outer stent base layer and the intermediate stent base layer, and the inner stent base layer and the intermediate stent base layer, even after decomposition of the biodegradable polymer in the first and second resinous adhesive layers.

13. The stent as defined in claim 8, wherein the inner stent base layer comprises a plurality of through holes extending from an inner surface of the inner stent base layer to the second resinous adhesive layer.

14. The stent as defined in claim 8, wherein the outer stent base layer comprises a plurality of through holes extending from an outer surface of the outer stent base layer to the first resinous adhesive layer.

15. The stent as defined in claim 8, wherein the intermediate stent base layer has an outer surface layer and an inner surface layer, both of which are formed from metal, and also has a multilayered structure composed of resinous adhesive layers and metallic layers which are laminated alternately.

16. The stent as defined in claim 8, wherein the physiologically active substance is at least one species selected from the group consisting of anti-tumor agent, immunosuppressant, antibiotic, antirheumatic drug, antithrombotic drug, HMG-CoA reductase inhibitor, ACE inhibitor, calcium antagonist, antilipidemic agent, integrin inhibitor, antiallergic drug, antioxidant, GPIIbIIIa antagonist, retinoid, flavonoid, carotinoid, lipid metabolism improver, DNA synthesis inhibitor, tyrosine kinase inhibitor, antiplatelet drug, anti-inflammatory drug, tissue-derived biomaterial, interferon, and NO yield promoting substance.

17. The stent as defined claim 9, wherein the first physiologically active substance is at least one species selected from the group consisting of anti-tumor agent, immunosuppressant, retinoid, flavonoid, DNA synthesis inhibitor, and tyrosine kinase inhibitor, and the second physiologically active substance is at least one species selected from the group consisting of antibiotic, antirheumatic drug, antithrombotic drug, HMG-CoA reductase inhibitor, ACE inhibitor, calcium antagonist, antilipidemic agent, integrin inhibitor, antiallergic drug, antioxidant, GPIIbIIIa antagonist, carotinoid, lipid metabolism improver, antiplatelet drug, anti-inflammatory drug, tissue-derived biomaterial, interferon, and NO yield promoting substance.

18. The stent as defined in claims 1, wherein the stent is formed in a tubular shape, has a diameter small enough for insertion into a lumen of a living body, and is capable of expansion upon application of an outward force in a radial direction from its inside.

19. A stent delivery device comprising:
a tubular shaft body comprising a tip;
a collapsible and expandable balloon attached to the tip of the tubular shaft body;
the stent defined in claim 18; and
the stent surrounding the balloon in a collapsed state and expandable upon expansion of the balloon.
